(19) United States
(12) Patent Application Publication
(10) Pub. No.: US 2004/0247073 A1
(43) Pub. Date: Dec. 9, 2004

(54) High resolution X-ray system

(76) Inventors: Yong Min Cho, Silver Spring, MD (US); Daesoo Han, Silver Spring, MD (US)

Correspondence Address: HARNESS, DICKEY & PIERCE P.L.C., P O BOX 8910, RESTON, VA, 20195, US

(21) Appl. No.: 10/452,508

(22) Filed: Jun. 3, 2003

(51) Int. Cl. G21K 7/00

(52) U.S. Cl. 378/43

(57) ABSTRACT

Every year numerous people are diagnosed with cancer, many of whom are detected in the latter stages of the cancers' progression. The later the cancer is detected, the more difficult it becomes to treat the cancer. Due to inefficient technology and late diagnosises, many people lose their lives to cancer each year. If this new X-ray system is implemented and put into use by medical doctors, not only will tumors be detected earlier, but smaller tumors, which are often overlooked by conventional X-ray systems, will also be detected. In addition, due to the multiple angles provided by this system, the exact location of these cancerous tumors can be pinpointed, thereby saving time that is currently wasted in determining exact tumor locations and allowing the patient to go into surgery sooner to have the malignant tumor removed. Thus, the development and implementation of this new technology will save many cancer patients' lives through early and more precise diagnosises.

### BACKGROUND OF THE INVENTION

For many years the NASA Astrophysics Group has used X-ray telescopes to detect and measure the X-rays emitted from the space. When we reverse the processes of the X-ray telescope in front of a medical X-ray machine, with a high resolution X-ray detector array, we can create a high resolution X-ray system with parallel X-ray beams of uniform density. This X-ray system will give more precise real-time X-ray information to medical doctors. Also, since the system uses parallel X-ray beams, both the amount of power required to work the system and the patient's total radiation exposure can be reduced. This system also has the advantage of time efficiency, as it would quickly scan an entire body in comparison to CAT scans. It will be the revolutionary X-ray machine for the medical world. It will be very useful to detect small tumors in the body and help people to detect cancer in its early stages. This advantage will save many human lives from cancer.

### SUMMARY OF THE INVENTION

The primary objective of this invention is to provide a uniform density X-ray beam to an X-ray detector system, which will be able to provide accurate X-ray derived images of the human body for medical purposes. This new X-ray technology will provide precise images using low power and minimizing the patient's exposure to radiation, thereby making it the most efficient X-ray system.

### BRIEF DESCRIPTION OF THE DRAWING

This invention can be fully understood through references to the following two figures and their brief descriptions below:

FIG. 1 is a conceptual view of the entire high resolution X-ray system.

FIG. 2 is a conceptual view of the X-ray system used from two angles.

### DETAILED DESCRIPTION OF THE DRAWING

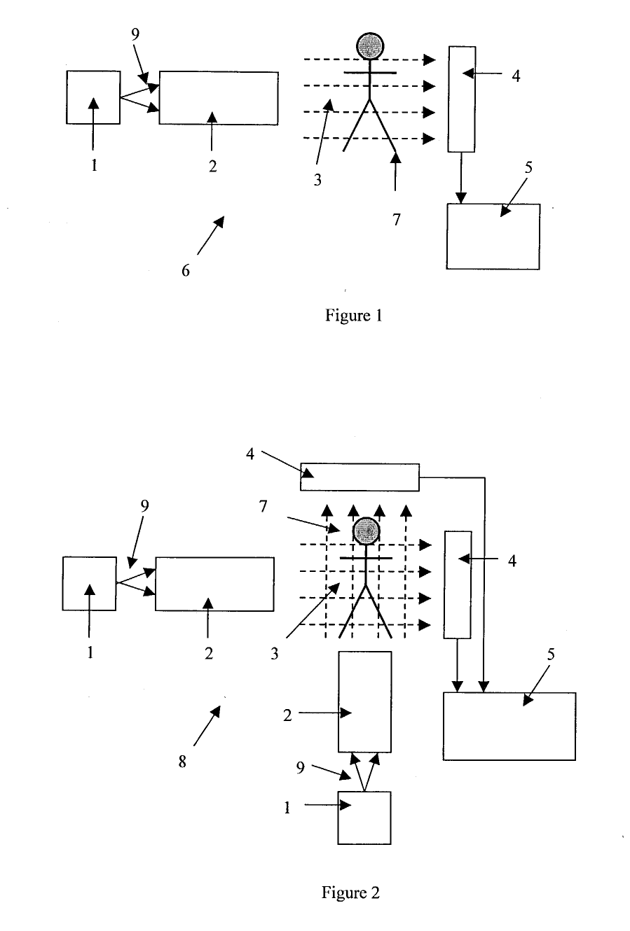

In FIG. 1 the X-ray system 6 is comprised of an X-ray emitter 1 that emits X-rays 9, which are passed through the X-ray telescope 2. The X-ray telescope 2 converts the X-rays 9 into parallel beams of uniform density 3 and passes them through the patient 7. After the beams 3 have passed through the patient 7, they are detected by the X-ray detector array 4. The detector array 4 relays the information it receives from the X-rays to a data processor 5, which compiles a composite image of the patient's body and reveals the existence and location of tumors.

In FIG. 2 two X-ray systems 8 are being used together to provide a more accurate image from two different angles. The X-ray emitter 1 emits X-rays 9 that are converted into parallel beams of uniform density 3 by the X-ray telescope 2. These beams 3 pass through the human body 7 and are read by the X-ray detector array 4. The information is processed by the data processor 5, which compiles a composite image of the patient's body from two different angles.

I claim:

1. An medical X-ray system that uses a reversed X-ray telescope in conjunction with an X-ray emitter to create parallel X-ray beams of uniform density.
